(19) United States
(12) Patent Application Publication
(10) Pub. No.: US 2024/0404630 A1
(43) Pub. Date: Dec. 5, 2024

(54) SYSTEMS AND METHODS FOR SECURE GENOMIC ANALYSIS USING A SPECIALIZED EDGE COMPUTING DEVICE

(71) Applicant: HAYSTACKANALYT ICS PVT. LTD., Bangalore, Karnataka (IN)

(72) Inventors: Anirvan CHATTERJEE, Mumbai,Maharashtra (IN); Priyanku KONAR, Mumbai,Maharashtra (IN); Sanchi SHAH, Mumbai,Maharashtra (IN); Sanjana KURUWA, Mumbai,Maharashtra (IN)

(21) Appl. No.: 18/700,485

(22) PCT Filed: Oct. 13, 2022
(86) PCT No.: PCT/IN2022/050915

§ 371 Date: Apr. 11, 2024

(30) Foreign Application Priority Data
Oct. 13, 2021 (IN) 202121046681

(51) Int. Cl. G16B 20/20; G16B 30/20; G16B 50/30

(52) U.S. Cl. CPC G16B 20/20; G16B 30/20; G16B 50/30

(57) ABSTRACT

Embodiments herein disclose systems and methods for secure genomic analysis using a specialized edge computing device (10). The edge computing device (10) can access a genomic platform (30) that enables a genomic analysis unit (12) inside the edge computing device (10) to perform genomic analysis of an input sequence data of a sample. The genomic analysis that is performed may be based on a selection by a user of the edge computing device (10). The genomic analysis unit (12/22) outputs a report comprising details of the genomic analysis of the input sequence data.

### CROSS REFERENCE TO RELATED APPLICATION

This application is based on and derives the benefit of Indian Provisional Application No. 202121046681, the contents of which are incorporated herein by reference.

### TECHNICAL FIELD

Embodiments disclosed herein relate to genomic data analysis, and more particularly to a system and method for secure genomic data analysis using a specialized edge computing device.

### BACKGROUND

Genetic data analysis is now paramount in various aspects of biology such as drug development, as well as other major decisions involved in areas of healthcare and general biological research such as clinical trials, environmental studies, evolutionary studies, etc. In the case of infectious diseases, especially with pandemics, it becomes highly beneficial that the processes of drug discovery are simplified. As the genomic data generated through newer sequencing methods are extensive, it can make genomic analysis computationally intensive. Genomics also has several applications in non-communicable diseases such as cardiovascular disease, cancer or rare disease diagnosis as well as evaluating the most appropriate therapy for an individual. Genomics is an important avenue for personalized medicine.

At present, genomic analysis may require a multidisciplinary team to analyze the data further in a meaningful way which can make it resource-intensive and time-consuming. Being computationally intensive makes the current day methods non-scalable and non-standardized. Traditional solutions to automate the genomic analysis process have not been successful as only parts of the genomic analysis have been automated, thereby these traditional solutions do not provide a complete solution to the resource and time-intensive process. Furthermore, the present-day solutions may require an expert bioinformatician to perform the analysis or may require specialized computational power. Currently existing applications for genomic data analysis are only done in limited laboratories or through a few tools for specific tests. While bioinformatic analysis for genomic data can be automated for use by diagnostic lab users who are not experienced in bioinformatics, the drawback is that bioinformatics also requires high computation capacity and infrastructure that is not widely available. In such cases standardized workflows may not be implemented and yield varying results dependent on the team performing the analysis. Implementing an automated solution could result in error-free and standardized analysis.

While there have been some solutions specific only for a particular disease and diagnostic analysis, the drawback is that these solutions may not be applicable for multiple different genomic analyses. For cloud-based solutions for genomic analysis, the sequenced data can yield large files (a few GB in size) and uploading the same to the cloud may require a highspeed internet connection. Moreover, it is desirable to ensure security of the sequenced data by limiting its access to authorized entities.

A significant challenge when it comes to genomic analysis for various types of samples is the reproducibility of the data, and also providing a clinician with the required information for taking an action. For example, if an analysis to determine the drug resistance profile of a bacteria present in an individual was performed, then based on an output which lists out the various genomic signatures that relate to antibiotics that the bacteria is resistant to, a clinician would know what antibiotics to administer and prescribe to the individual to eliminate the bacteria.

Some of the aforementioned problems with the existing solutions to analyze genomic data are that it is time-consuming and costly, owing to which these solutions are an impedance to its scalability and the areas of usage (e.g., these solutions may only be limited to research purposes). Some existing solutions relate to only the analysis of specific next-generation sequencing (NGS) data or can only be used with a specific sequencing platform. Some other existing solutions only provide analysis only for a specific disease condition or a specific analysis that may not be easily updated as scientific knowledge progresses. Such solutions become redundant in a short period and a new analysis needs to be built on new scientific information. Accordingly, it is desirable to implement a system or platform for genomic analysis that overcomes the aforementioned technical drawbacks in existing technologies by providing scalable and cost-effective genomic analysis solutions for healthcare-related decision-making.

### Objects

The principal object of embodiments herein is to disclose a system and method for secure genomic data analysis using a specialized edge computing device.

These and other aspects of the embodiments herein will be better appreciated and understood when considered in conjunction with the following description and the accompanying drawings. It should be understood, however, that the following descriptions, while indicating at least one embodiment and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

Embodiments herein are illustrated in the accompanying drawings, throughout which like reference letters indicate corresponding parts in the various figures. The embodiments herein will be better understood from the following description with reference to the drawings, in which:

FIG. 1 illustrates a system comprising an edge computing device for performing secure genomic analysis, according to embodiments as disclosed herein;

FIG. 2 illustrates the features of the user interface of the edge computing device, according to embodiments as disclosed herein;

FIG. 3 illustrates the various modules in the genomic analysis unit, according to embodiments as disclosed herein;

FIG. 4 illustrates the services offered by the private cloud server, according to embodiments as disclosed herein;

FIG. 5 illustrates a method for performing the genomic data analysis, according to embodiments as disclosed herein;

FIGS. 6A-6B illustrate a method for determining the drug resistance profile of a sample having tuberculosis, according to embodiments as disclosed herein; and

FIGS. 7A-7B illustrate a sample tuberculosis report pertaining to the drug resistance of a sample having tuberculosis, according to embodiments as disclosed herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein disclose systems and methods for performing secure genomic analysis using an edge computing device. The edge computing device may externally receive raw genomic data (the genomic data that is to be analyzed/interpreted) and metadata, and perform genomic analysis of that raw genomic data (also referred to as “input sequence data” and “genome sequence data”). The final output, which maybe through the edge computing device, can be a report that includes the genomic analysis of the input sequence data. The report can provide a clinician with clear actionable information in a short span of time that will allow the clinician to provide an individual with the appropriate treatment. The edge computing device may access a genomic platform that enables the edge computing device to perform the genomic analysis.

The embodiments herein provide convenience to an end user by streamlining the process of obtaining an analysis report of the genomic data by having an edge computing device that can perform the genomic analysis in an automated manner without requiring any human input or monitoring, or any specialized expertise on the end user's part. The embodiments work in a self-orchestrated manner where upon a single-click (choosing of the analysis to be performed) by the end user, an analysis report comprising the genomic analysis of the input sequence data is generated.

As the genomic analysis may be performed on the edge computing device side, there is no need to upload large data files to any remote platform, due to which the genomic analysis process on the edge computing device side is faster, more cost-effective, and secure. In some embodiments, the system comprises a hybrid computational model, wherein one portion of the genomic analysis is performed by the edge computing device, and another portion is performed on a server side. The genomic analysis of the raw genomic data may be performed for a specific use case that is selected by a user or selected automatically. The analysis report generated may have a quicker turnaround time compared to an analysis performed by a lab technician.

The embodiments herein may use dynamic orchestration, wherein on the user selecting (or an automatic selection) the analysis to be performed on the input sequence data, the system decides the best manner in which the selected analysis is to be performed. The embodiments disclosed herein use a modular and flexible approach, where the sequenced data generated from sequencing (e.g., short or long read sequencing) across various sequencing platforms may be accepted. The embodiments herein disclose a genomic analysis process that accepts the sequenced data, recognizes the sequenced data, and then streamlines it to run through various modules for genomic analysis. Each module in the embodiments disclosed herein can generate an output that may be taken as an input by another module. For example, in a situation where binning of tuberculosis data containing coinfection is done, one module may analyze the tuberculosis data while another module may analyze the non-tuberculosis data. In the embodiments disclosed herein, the input and output data for each module may be standardized. The final output of the genomic analysis process can be a standardized clinically relevant report.

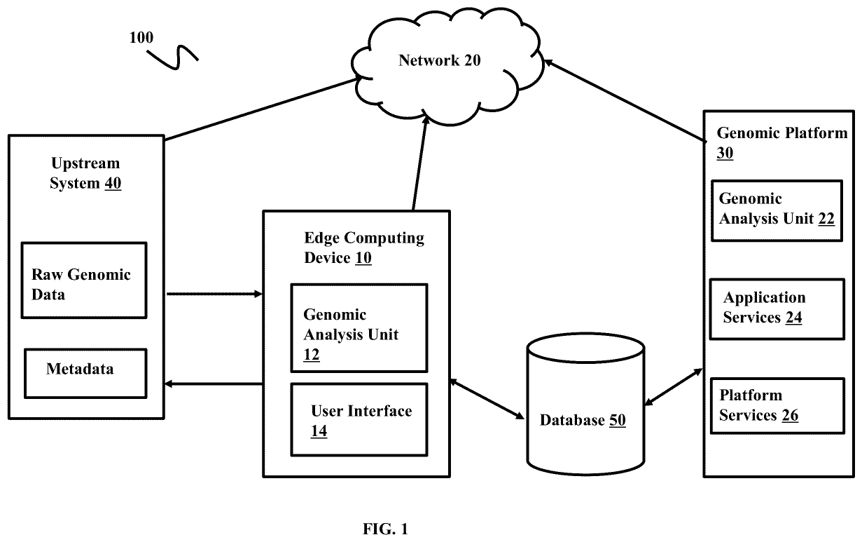

Referring now to the drawings, and more particularly to FIGS. 1 through 7, where similar reference characters denote corresponding features consistently throughout the figures, there are shown embodiments.

FIG. 1 illustrates a system 100 comprising an edge computing device 10 and a genomic platform 30 for performing the genomic analysis, according to embodiments as disclosed herein. As will be understood by one of ordinary skill in the art, these systems and methods may be implemented in any suitable way.

The upstream system 40 can include an entity that collects raw genomic data and/or metadata. Examples of the upstream system 40 can be a sequencing lab, a laboratory information management system (LIMS), or a hospital management system (HMS).

The network 20 can include a variety of types of computer networks, such as, but not limited to, the Internet, a private intranet, a mesh network, or any other type of network. The edge computing device 10 can communicate with the upstream system 140 and the genomic platform 30 using the network 20 to receive and/or transmit data.

The edge computing device 10 may be a personal computer (PC), a tablet, or a smartphone, but is not limited to this. The edge computing device 10 may interact with the genomic platform 30 through means such as, but not limited to, a browser or a desktop GUI application that is installed or embedded in the device 10. Upon accessing the platform 30 through the website and/or the application, the platform 30 can provide a user of the device 10 with a user interface 14. In some embodiments, the edge computing device 10 may be provided to a user (provided with or without peripheral devices such as a keyboard etc.), wherein the computing device 10 is a specialized device having the application embedded in it.

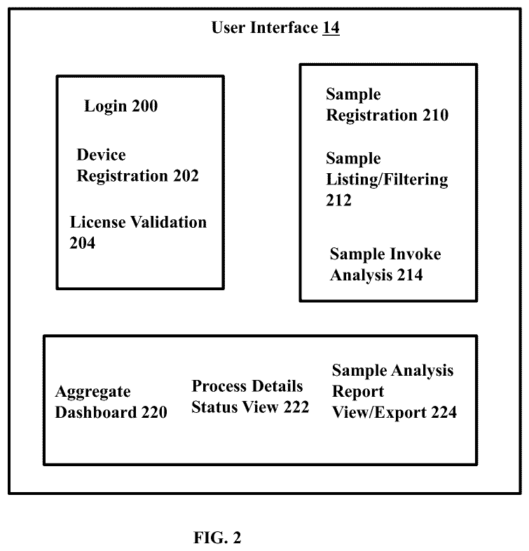

As illustrated in FIG. 2, the user interface 14 illustrates the various features of and services offered by the platform 30. Examples of the various features and services include, but are not limited to, a login section 200, a device registration section 202, a license validation section 204. The user interface 14 further comprises a sample registration section 210, a sample listing/filtering section 212, and a sample invoke analysis section 214. The user interface further comprises an aggregate dashboard section 220, a process details status view section 222, and a section 224 for viewing or exporting the report including the sample analysis.

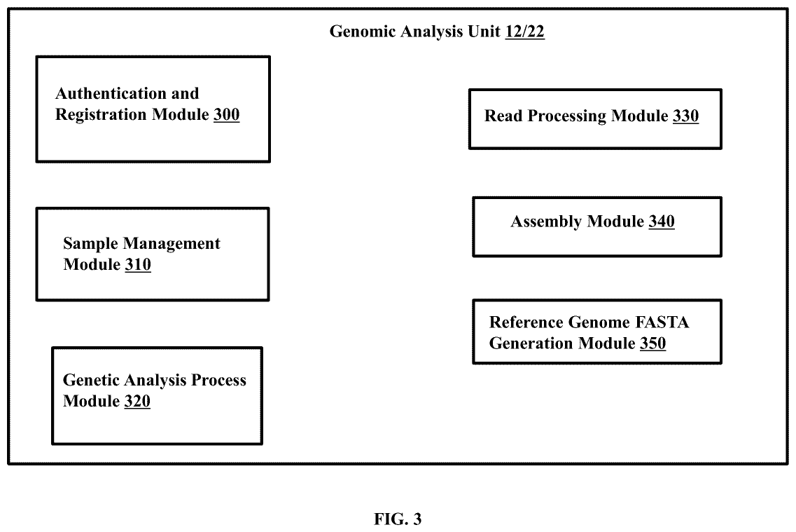

As illustrated in FIG. 3, a genomic analysis unit 12 of the edge computing device 10 can include a plurality of modules that enables the edge computing device 10 to perform the features and services displayed in the user interface 14. The authentication and registration module 300 allows for a user to log in to the platform 30, register their device 10 with the platform 30, and validate a license that they obtained to use the platform 30. This ensures secured genomic analysis as only selected devices 10 would be able to access the platform 30. The authentication process can involve two-factor authentication or multifactor authentication. In some embodiments, the two-factor authentication may include the use of methods such as short messaging service (SMS), authenticator generation applications, push messages, and other methods known in the art.

The sample management module 310 can allow for a user to register a sample, list or filter samples, and invoke a certain analysis for the registered sample.

The genetic analysis process module 320 may perform the analysis of the raw genomic data, provide details of the genomic analysis being performed and the current status of the analysis. The module 320 may also be responsible for generating the report including the genomic analysis, wherein the report may be viewed and exported.

The read processing module 330 may be responsible for read mapping, read binning, decontamination and read mapping to bins.

The assembly module 340 may be processing for performing de novo assembly.

The reference genome FASTA generation module 350 may be responsible for creating a reference genome for comparison with the sequenced data of a sample to bin the non-relevant data.

The genomic analysis unit 12/22 may comprise at least one processor that is configured to perform the functions associated with the modules present in it. It is to be noted that FIG. 3 illustrates a non-exhaustive list of modules present in the genomic analysis unit 12/22.

The user interface 14 allows for a user to provide several inputs, such as the type of genomic analysis to be performed for the raw genomic data. The edge computing device 10 may communicate with the database 50 that may be configured to store details such as the user's login credentials, the analysis reports generated etc. The database 50 may be a part of the genomic platform 30 itself.

The genomic platform 30 may operate as a physical or virtual server, which can include but is not limited to, a web server, an application server, a cloud server, or a database server. The platform 30 can comprise an application service layer, a storage layer, a high-performance computing layer, and a process orchestrator engine. The platform 30 can facilitate the analysis of the input sequence data on the edge computing device 10. In some embodiments, the platform 30 can include a genomic analysis unit 22, which can function similarly to its counterpart 12 in the edge computing device 10, wherein a portion of the genomic analysis is performed on the edge computing device 10 and the remaining portion is performed on the platform 30.

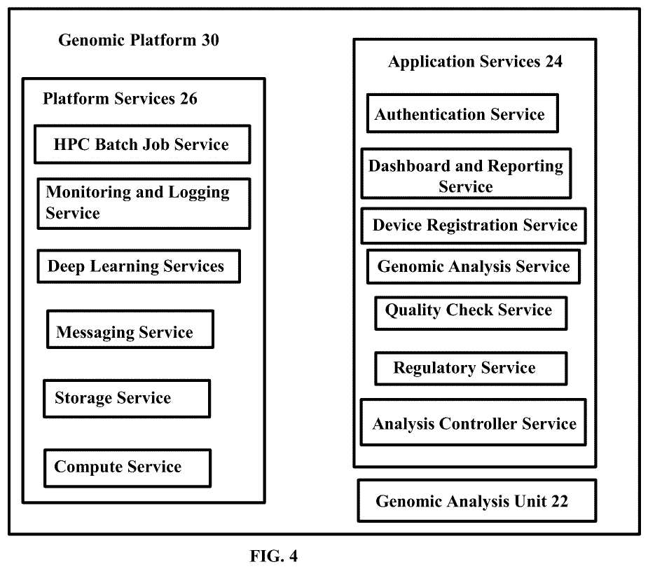

As illustrated in FIG. 4, the application services 24 include application programming interface (API) services that allow the edge computing device 10 to interact with the platform 30. Examples of the application services 24 include quality check service, regulatory service, and analysis controller service.

The platform services 26 the services that can be used to create the application services. Examples of the platform services 26 include high performance computing (HPC) batch job service, monitoring and logging service, deep learning services, messaging service, storage service, and compute service.

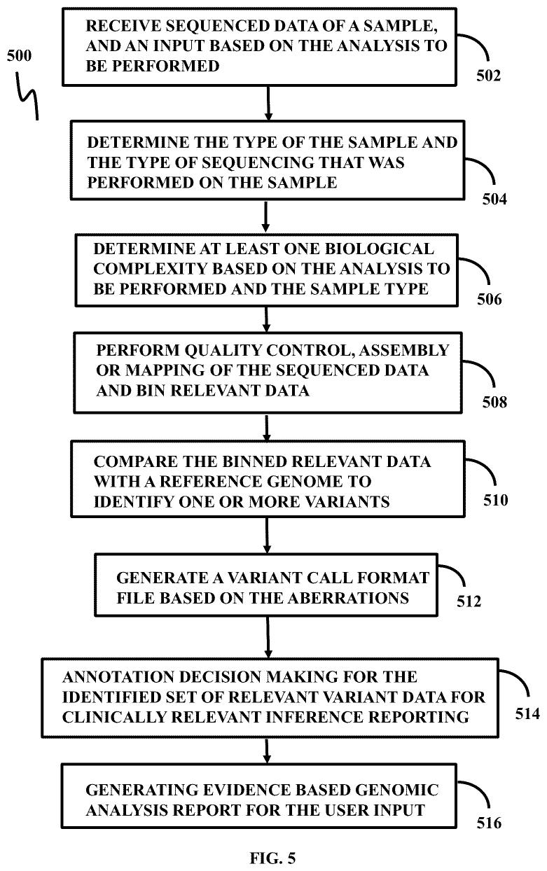

FIG. 5 illustrates a method 500 for performing the genomic analysis, according to embodiments as disclosed herein.

At step 502, the genomic analysis unit 12/22 may receive the sequenced data of a sample, and an input (user-selected input or automated selection) regarding the analysis to be performed on the sequenced data of the sample. The sample can be sterile body fluids, non-sterile body fluids, germline sample, somatic sample, or cell free DNA samples.

At step 504, the genomic analysis unit 12/22 may determine the type of sample and the type of sequencing (e.g., short read sequencing, long read sequencing, shotgun sequencing, whole genome sequencing, targeted sequencing etc.) that was performed on the sample.

At step 506, the genomic analysis unit 12/22 may determine at least one biological complexity based on the analysis to be performed and the sample type. Examples of the biological complexity can be the presence of a co-infection for drug resistance profile, or a strain level variation for pathogen detection etc.

At step 508, based on the at least one biological complexity and the type of sequencing that was performed, the genomic analysis unit 12/22 may perform quality control of the sequenced data, and then perform assembly/mapping, which then results in the remaining data being the relevant data that is binned. The quality control of the sequenced data can result in filtering out data that does not pass a quality score threshold (e.g Phred score), high error rates etc. For example, for long read sequencing, the sequenced data may be prone to high error rates, due to which such data would need to be filtered out.

If short read sequencing is performed and the at least one biological complexity is the presence of a coinfection, then the sequenced data may be checked to see if there was an adequate depth of sequencing across every mutation in the sequenced data by comparing the sequenced data with a catalogue of mutations in a relevant genome for a relevant case. For example, to determine a drug resistance profile of tuberculosis, the sequenced data of a tuberculosis sample may be compared with a catalogue of mutations in a tuberculosis genome (relevant genome) that is associated with drug resistance (relevant case).

Based on the determination of whether there was an adequate depth of sequencing, it can be understood if the sequenced data of the sample was sequenced properly or not. It can also be understood from the sequenced data if the sequenced data predominantly includes coinfections or not.
a. If the sequenced data does not include any contamination or coinfection, then the entire sequenced data may be considered as relevant, and it is then analyzed.
b. If the sequenced data includes some contamination or coinfection that is not predominant, then the relevant genome data is binned for further analysis, while the non-relevant genome undergoes de novo assembly, which can be used for reporting the coinfection.
c. If the sequenced data is predominantly contaminated or comprising the coinfection, then the entire sequenced data may undergo de novo assembly, and may then be compared with a reference genome (which may be created by the module 350 and in a FASTA format) to distinguish the data of the relevant genome from the non-relevant data genome (data about contaminations/coinfections). The relevant genome data may be analyzed, while the non-relevant genome data is binned and may be used for reporting coinfections. This reporting of coinfections can be done by creating FASTAs of the non-relevant genome data.

At step 510, the genomic analysis unit 12/22 may compare the binned relevant data with a reference genome to obtain identity one or more variants, and thereby obtain the aberrations. The reference genome may be present in a catalogue that is accessible to the genomic analysis unit 12/22 via the database 50.

At step 512, the genomic analysis unit 12/22 may generate a variant call format file based on the aberrations.

At step 514, the genomic analysis unit 12/22 may annotate those aberrations that are relevant/significant (evidence-based aberrations). Certain variants may be known to have a particular implication, based on which the annotations are made.

At step 516, the genomic analysis unit 12/22 may generate an evidence-based genomic analysis report for the analysis that was performed on the sequenced data of the sample. This analysis report may be viewed by the user on the device 10 or exported as a file. In some embodiments, this analysis report may be transmitted to the upstream systems 40, such as LIMS or HMS. The generated report may include actionable information that enables a clinician to know how to proceed with treatment for an individual.

The various steps in method 500 may be performed in the order presented, in a different order or simultaneously. Further, in some embodiments, some steps listed in FIG. 5 may be omitted or some steps may be added. The genomic analysis unit 12/22 may comprise a plurality of modules (in addition to the modules listed in FIG. 3) to perform the steps in the method 500.

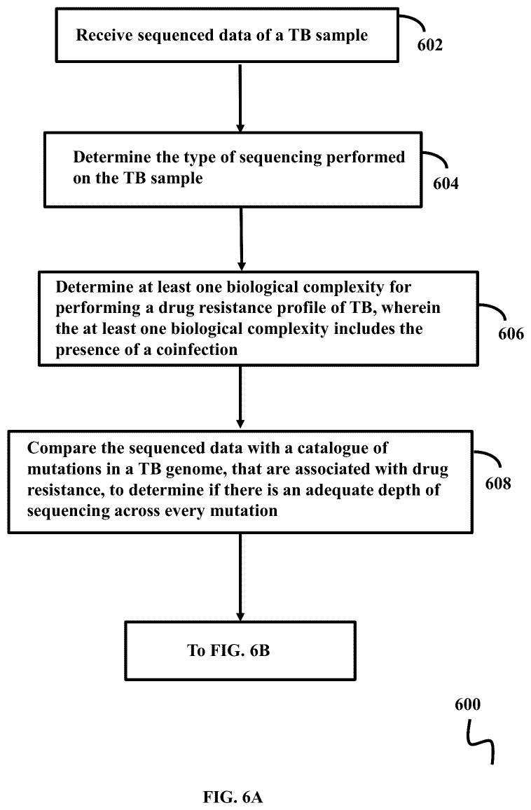

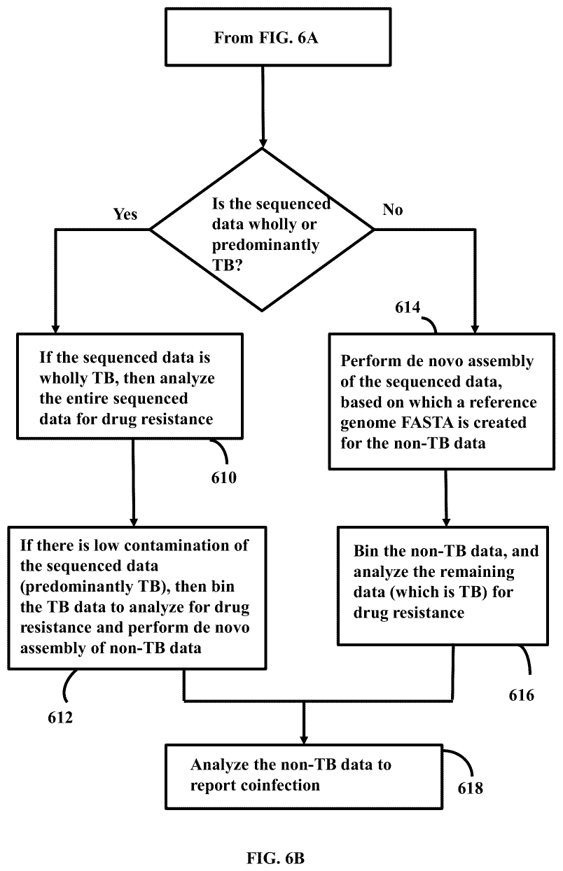

FIGS. 6A to 6B illustrates a method 600 for determining the drug resistance profile of a tuberculosis (TB) sample, by the genomic analysis unit 12/22, according to embodiments as disclosed herein. The TB sample can be sputum or other body tissue samples.

At step 602, the genomic analysis unit 12/22 may receive the sequenced data of the TB sample having the TB bacteria.

At step 604, the type of sequencing performed on the TB sample may be determined by the genomic analysis unit 12/22. For short read sequencing of the TB sample, the short read sequencing data can employ different parameters for assembly, mapping, and quality control. Non-exclusive and non-limiting examples of these parameters include read length with 75-250 base pairs, insert length of 100-300 base pairs, Phred score that is greater than 20, a depth of sequencing at 30 times, allelic discrimination of less than 10%, and PCR duplicates greater than 5. For other types of sequencing, there may be different parameters.

At step 606, the genomic analysis unit 12/22 may determine at least one biological complexity for performing a drug resistance profile of TB, wherein one of the biological complexities includes the presence of coinfections.

At step 608, the genomic analysis unit 12/22 may compare the sequenced data with a catalogue of mutations in a TB genome, that are associated with drug resistance, to determine if there is an adequate depth of sequencing across every mutation. This can help determine the presence of other infections alongside TB.

If the sequenced data is wholly TB, then at step 610, the entire sequenced data may be analyzed for drug resistance.

If the sequenced data is predominantly TB, then at step 612, the TB data may be binned for drug resistance analysis, while the non-TB data may undergo de novo assembly.

If the sequenced data is not predominantly TB, then at step 614, the entire sequenced data may undergo de novo assembly. A reference genome (in FASTA format) can be created to distinguish between the TB data and non-TB data in the sequenced data. At step 616, the non-TB data may be binned, while the remaining data (TB data) is analyzed for drug resistance.

At step 618, the non-TB data in steps 612/616 may be used for reporting coinfections.

The various steps in method 600 may be performed in the order presented, in a different order or simultaneously. Further, in some embodiments, some steps listed in FIGS. 6A-6B may be omitted. The genomic analysis unit 12/22 may comprise a plurality of modules (in addition to the modules listed in FIG. 3) to perform the steps in the method 600.

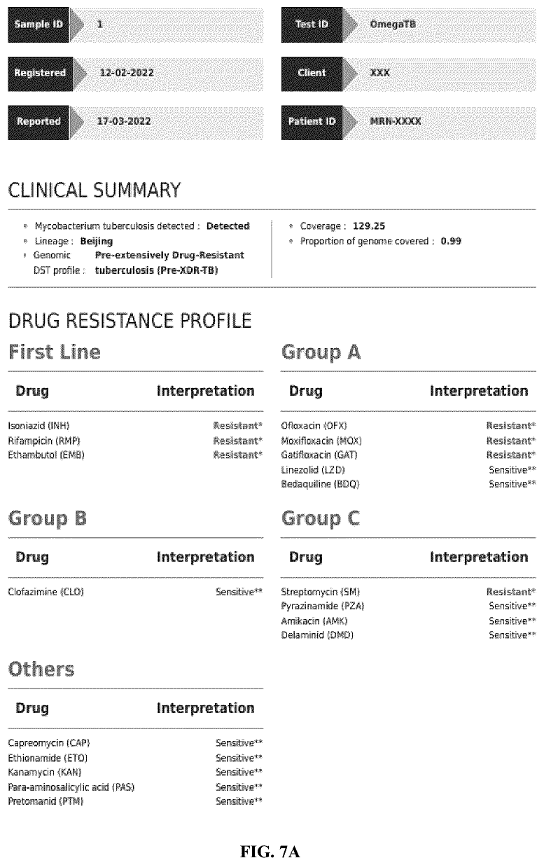

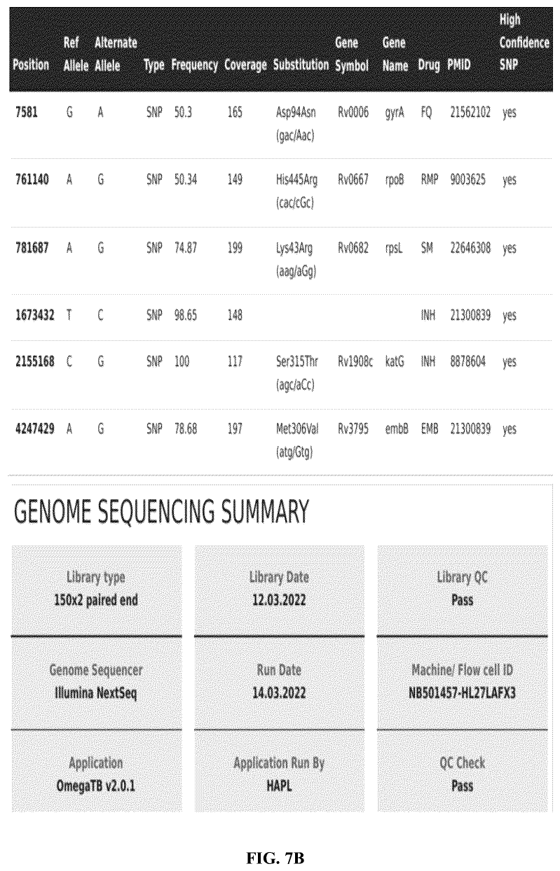

FIGS. 7A-7B illustrate a sample report of drug resistance profile of TB based on the performance of method 600, according to embodiments as disclosed herein. FIG. 7A illustrates the clinical summary and the list of drugs that the TB is resistant or sensitive to. FIG. 7B illustrates a mutation table of the TB sample. The details of a tuberculosis report that is generated from performing method 600 can include details such as, but not limited to, strain identification and drug resistance markers that may be done based on single nucleotide polymorphism (SNP), insertion-deletions etc. calling of neutral genomic markers, multidrug resistant (MDR), pre-extensive drug resistant (Pre-XDR), extensively drug resistant (XDR), non-tuberculous-mycobateria, coinfections, drug resistance profile based World Health Organization (WHO) drug group, and a mutation table with depth, coverage, mutation, amino acid change, validation study reference and confidence of mutation.

In some of the embodiments disclosed herein, the genomic analysis may be wholly performed on the edge computing device 10 or the genomic platform 30; in other embodiments, the genomic analysis may be performed in a hybrid model where the edge computing device 10 performs some steps of methods 500/600 while the genomic platform 30 performs the remaining steps of methods 500/600. The system 100 comprises the edge computing device 10 and the genomic platform 30 that may each comprise a memory and at least one processor 12/22. The at least one processor 12/22 may be coupled to the memory, wherein the at least one processor 12/22 is configured to perform the steps of methods 500 and 600. The memory may comprise, but is not limited to, volatile (e.g. random access memory (RAM)), non-volatile (e.g. read-only memory (ROM)), flash memory, or any combination. The at least one processor 12/22 represents one or more processors such as a microprocessor, a central processing unit or the like. The at least one processor 12/22 may also be a special-purpose processor such as an application specific integrated circuit (ASIC), a field programmable gate array (FPGA), a digital signal processor (DSP), a network processor, or the like.

The embodiments disclosed herein can be implemented through at least one software program running on at least one hardware device and performing network management functions to control the network elements. The network elements shown in FIG. 1 include blocks which can be at least one of a hardware device, or a combination of hardware device and software module.

The embodiment disclosed herein describe a system and method for performing secure genomic analysis using an edge computing device 10. Therefore, it is understood that the scope of the protection is extended to such a program and in addition to a computer readable means having a message therein, such computer readable storage means contain program code means for implementation of one or more steps of the method, when the program runs on a server or mobile device or any suitable programmable device. The method is implemented in at least one embodiment through or together with a software program written in e.g. Very high speed integrated circuit Hardware Description Language (VHDL) another programming language, or implemented by one or more VHDL or several software modules being executed on at least one hardware device. The hardware device can be any kind of portable device that can be programmed. The device may also include means which could be e.g. hardware means like e.g. an ASIC, or a combination of hardware and software means, e.g. an ASIC and an FPGA, or at least one microprocessor and at least one memory with software modules located therein. The method embodiments described herein could be implemented partly in hardware and partly in software. Alternatively, the invention may be implemented on different hardware devices, e.g. using a plurality of CPUs.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments and examples, those skilled in the art will recognize that the embodiments and examples disclosed herein can be practiced with modification within the spirit and scope of the embodiments as described herein.

1. A method (500) for performing a genomic analysis, comprising:
receiving, by a genomic analysis unit (12/22), a sequenced data of a sample, and an input based on the type of the genomic analysis to be performed on the sequenced data;
determining, by the genomic analysis unit (12/22), the type of the sample and the type of sequencing that was performed on the sample;
performing, by the genomic analysis unit (12/22), quality control, assembly or mapping of the sequenced data, upon which data, that is relevant to the genomic analysis type, is obtained and binned;
comparing, by the genomic analysis unit (12/22), the relevant data with a reference genome to identify one or more variants, upon which a plurality of aberrations are obtained;
generating, by the genomic analysis unit (12/22), a variant call format file based on the plurality of aberrations; and
annotating, by the genomic analysis unit (12/22), those aberrations, among the plurality of aberrations, that are relevant to the genomic analysis type.

2. The method (500) of claim 1, further comprising:
determining, by the genomic analysis unit (12/22), at least one biological complexity based on the genomic analysis type and the type of the sample;
generating, by the genomic analysis unit (12/22), a report comprising details of the genomic analysis performed, wherein the details are based on the relevant aberrations.

3. The method (500) of claim 1, wherein if the sequencing type was short read sequencing and the at least one biological complexity includes the presence of a coinfection, then the quality control involves the following:
determining if there is an adequate depth of sequencing across every mutation in the sequenced data by comparing the sequenced data with a list of mutations in a relevant genome that is relevant to the genomic analysis type;
based on the determination of adequate depth of sequencing, performing one of the following:
analyzing the sequenced data, in its entirety, if it is wholly relevant;
binning the portion of the sequenced data that is relevant (relevant data) for analysis, and performing de novo assembly of a non-relevant portion of the sequenced data (non-relevant data); and
performing de novo assembly of the sequenced data in its entirety, filtering out the non-relevant data by comparing it with a second reference genome, binning the non-relevant data, and analyzing the relevant data.

4. A method (600) for determining the drug resistance of a sample having tuberculosis (TB), comprising:
receiving, by a genomic analysis unit (12/22), a sequenced data of the TB sample;
determining, by the genomic analysis unit (12/22), the type sequencing that was performed on the TB sample;
comparing, by the genomic analysis unit (12/22), the sequenced data with a catalogue of mutations in a TB genome, that are associated with drug resistance, to determine if there is an adequate depth of sequencing across every mutation in the sequenced data; and
analyzing, by the genomic analysis unit (12/22), the drug resistance of the portion of the sequenced data that corresponds to TB (TB data), wherein the analysis is a determination of the drug resistance of the TB in the sample.

5. The method (600) of claim 4, further comprising:
determining, by the genomic analysis unit (12/22), at least one biological complexity based on the type of the TB sample, wherein the at least one biological complexity includes the presence of at least one coinfection;
determining, by the genomic analysis unit (12/22), if the sequenced data is wholly, predominantly, or not predominantly including TB.

6. The method (600) of claim 5, wherein the sequenced data, in its entirety, is analyzed of drug resistance if the sequenced data wholly includes TB.

7. The method (600) of claim 5, wherein
the sequenced data, in its entirety, undergoes de novo assembly,
the portion of the sequenced data that does not correspond to TB (non-TB data) is filtered out by comparing the sequenced data with a reference genome, and the non-TB data is binned, and
analyzing the drug resistance of the TB data,
if the sequenced data is not predominantly including TB.

8. The method of claim 5, wherein
the TB data is binned for analysis of drug resistance, and
the non-TB data undergoes de novo assembly,
if the sequenced data predominantly includes TB.

9. The method (600) of claim 6, further comprising reporting, by the genomic analysis unit (12/22), the non-TB data for the presence of the at least one coinfection in the TB sample.

10. A system (100) for performing genomic analysis, comprising:
a memory storing a plurality of instructions; and
at least one processor (12/22) coupled to the memory, wherein the at least one processor (12/22) is configured to execute the plurality of instructions to perform the following:
receiving a sequenced data of a sample, and an input based on the type of the genomic analysis to be performed on the sequenced data;
determining the type of the sample and the type of sequencing that was performed on the sample;
performing quality control, assembly or mapping of the sequenced data, upon which data, that is relevant to the genomic analysis type, is obtained and binned;
comparing the relevant data with a reference genome to identify one or more variants, upon which a plurality of aberrations are obtained;
generating a variant call format file based on the plurality of aberrations; and
annotating those aberrations, among the plurality of aberrations, that are relevant to the genomic analysis type.

11. The system (100) of claim 10, wherein the at least one processor (12/22) executes the plurality of instructions to further perform the following:
determining at least one biological complexity based on the genomic analysis type and the type of the sample;
generating a report comprising details of the genomic analysis performed, wherein the details are based on the relevant aberrations.

12. The system (100) of claim 10, wherein if the sequencing type was short read sequencing and the at least one biological complexity includes the presence of a coinfection, then the quality control involves the following:
determining if there is an adequate depth of sequencing across every mutation in the sequenced data by comparing the sequenced data with a list of mutations in a relevant genome that is relevant to the genomic analysis type;
based on the determination of adequate depth of sequencing, performing one of the following:
analyzing the sequenced data, in its entirety, if it is wholly relevant;
binning the portion of the sequenced data that is relevant (relevant data), and performing de novo assembly of a non-relevant portion of the sequenced data (non-relevant data); and
performing de novo assembly of the sequenced data in its entirety, filtering out the non-relevant data by comparing it with a second reference genome, binning the non-relevant data, and analyzing the relevant data.

13. The system (100) of claim 10, further comprising a user interface (14) that allows a user to provide the input on the type of the genomic analysis that is to be performed.

14. A system (100) for determining drug resistance of a sample including tuberculosis (TB), comprising:
a memory storing a plurality of instructions; and
at least one processor (12/22) coupled to the memory, wherein the at least one processor (12/22) is configured to execute the plurality of instructions to perform the following:
receiving, by a genomic analysis unit (12/22), a sequenced data of the TB sample;
determining, by the genomic analysis unit (12/22), the type sequencing that was performed on the TB sample;
comparing, by the genomic analysis unit (12/22), the sequenced data with a catalogue of mutations in a TB genome, that are associated with drug resistance, to determine if there is an adequate depth of sequencing across every mutation in the sequenced data; and
analyzing, by the genomic analysis unit (12/22), the drug resistance of the portion of the sequenced data that corresponds to TB (TB data), wherein the analysis is a determination of the drug resistance of the TB in the sample.

15. The system (100) of claim 14, wherein the processor (12/22) executes the plurality of instructions to further perform the following:
determining at least one biological complexity based on the type of the TB sample, wherein the at least one biological complexity includes the presence of a coinfection;
determining if the sequenced data is wholly, predominantly, or not predominantly including TB.

16. The system (100) of claim 15, wherein the sequenced data, in its entirety, is analyzed for drug resistance if the sequenced data wholly includes TB.

17. The system (100) of claim 15, wherein
the sequenced data, in its entirety, undergoes de novo assembly,
the portion of the sequenced data that does not correspond to TB (non-TB data) is filtered out by comparing the sequenced data with a reference genome, and the non-TB data is binned, and
analyzing the TB data for analysis of drug resistance,
if the sequenced data is not predominantly including TB.

18. The system (100) of claim 15, wherein
the TB data is binned for analysis of drug resistance, and
the non-TB data undergoes de novo assembly,
if the sequenced data predominantly includes TB.

19. The system (100) of claim 16, further comprising, reporting, by the at least one processor (12/22), the non-TB data for presence of at least one coinfection in the TB sample.
